SEQUENCE LISTING (6 sequences)

SEQ ID NO: 1 - DNA, 3702 bp, Sus scrofa
  CDS (49)..(279)
gacaagaaggtttccaatcagtccctgctatgtggatatttgttagcaatgactgatgtggaaactacatatgcagattttattgcttcaggaagaacaggtagaagaaatgcaatacatgatatcctggtttcctctgcaagtggcaacagcaatgaattagccttgaaattagcaggtcttgatatcaacaagacagaaggtgaagaggatgcacagcgaagttcgacagaacaaagtggggaagcccagggagaagcagcaaaatcagaaagctaacactccactttgatctttatcaacacctgacaatgtctcaaatctccgggcctgtctggaatgcatttatttccgagagtgaaagggggaaaaagaaaatggctgttctgcattgccggaaactgcttgttatgttaaaaatgggggcagaggctgtggctgcagacagacttttctctacctctggcattagcaatggttgaaaatcatgtggcttgggtttggatgtcatttttgggatggatcctttcacttgaccatatatatgacgaaatgcttgtagagagtagccgcactgacctagatgatgattcttcctgtagcatctggcccctcacaatgtcagaggatttaattgtgtctaatcgcaaagggttggttgaaccccagagttttaaatatctctggcccaagtattcacccagtaaaagaaccatccagaaagcactgtttttaacattatgtatctgtgtgttcctgctgtgttatttacactgttttgtattgtacaatatagatgctcagcactgcccccttctttgattgcttatgaaaaacaaaaatgatgtcttgttactgtgaatttttataccactcatttttaaaagggctgcctttttttctgctccatagtgtggtggtgtacacaggataagatagaacacactttttttttttttaaacataatctttccccttaattcactgctgatgaattaagtctaacagattcatcaagactccttttgcttattgtacaggcattcgaaaatatccattaatgtgaatattacctgaattcagtctgtttggtgtctgcacagaccagaattcaatctgcaaattttgtcttttgcccaagtgtaaaactttttccaatgaatatttttattttcagttatgaggatttgggagaattggggaggtgggaaagagaatccaaggtaataccaagcactggaaatacttcccttctatgaaatcaaatttctcacagtgctgtatgatattattaaaatttggaggacaacttatctccacagagctgcaaaagatggagaaaatgaccttgcaatcatgtggacaccaatcacaaaaataaagcccttgtgtttttcatgtcttttttcagccccatcagatccaaatgttattatgcagtttttaatgtttgtaaacttttactaatataattagtgtgaattgcattctgatacaataataatcattattagaagctgacaaaaatccttattaatactgttgatggcctctgctgtgttttgacatcatggttcttatatggaaagtttcctgtgtaatccctccagtcagtattatgaaatcatttctcagtagtaataaaaaaggcaccagtaatatgcagtggctcatgaattactggataaatacaaggcaacaggaagaccctttacaataaaaagcccacttaactatcctcgaggtcttagatagcgtctcacgtgaagtaggttaggcagcatttcagcagataatcagtatggacaaaccaggatctcagccctcctttgtagttacataagtgatgaaaggtaagtctgccctcatctccctttacagtacattttgaatgagataagggcatgggtatcacttaatcctcaatcatgtattactagaaaaaatgttaactgtgtgactcttggagaaaataggaggaggaaagattaaattttagagaacttccttttgtctaacccaatagggtccaaaatggccagttaaatttcagaccaatccacctattaatagactcacaaatgggaaaagaggttcctgaggctattttagacaatttctggtagtccatttaggacatccacttacacatccatacaatcactttttcatatcctcatttattatcctcatagctttcttgtgcatccaggtaacattctcttcatccttaaatttatagagttccagactgatttcatgtgattctgtaaagtttaggactagtgctgagtatatgtggaggatttatattcctatgtgatatataattattaatgcatgtggtatcatgcttgtctttgaatatataatagtgctaaattgtgaagtcatatggagcttttgaattattttgacctcttactgctactttgtctgctatattcaaacccaaaggtattaccaggccagaaaataaaaatcaactctccaaaacatccacatcccatgaaagttgctcagcaaaagttcaaaataacttccaaattcctgtcatatcttttactttgcaagtcaataatatacatgggattgaatttttaaggagagaaagaaatgcatttggtttgattatacacattaaatgcctattaactgactttctgctaatcacttcagcacacatattcttccacttaatcctcaaacaattcaattaaagaggtaacattattccatttcaggaatcaggaaattaaagctcaataaggtataacttttacaagcttaaaaagctaataaatgatgaaataaaatttgaacctgtgtttatctgtctccagggagctttccacggcacatgctctaaaattgaagccagttacagagcatgtatgtctataagagatggactggatcagcctccatattttagagaaggaaagagtaattgttgatctcaaagttacctagatgagtaatgacgcagatgtgactagcacttttatttaaaaaaaataataataaatttaattaaaacttatttttttgtgttatactttccccagaagacatttttctgacatgaagtctataaacttagcatggttcagattttttacattccatgctactcattttgtttaattaactaggaatgtgataatttcagttttttgacttacttgcaataagagaaccttttcaaatttatctttaatcaattgtaagcatcctgtgctgatattgaaatcataaattcatagaaatgtttttacaacagatgacaaatgatttttaggcaatatccccagagctacacactgtaaatacccaacactcaagtcttacttacagtggaaatgtactgttttagaagatgaacaatattgcttcagagaggcaataaggggttaaaaaaaaaaaatgtaactgtgggctgtaaagtttcttggtgaagccagtagtcatggggagaatatgtaaagtcatcaagtgaaaacaggaaaggaactatgtttatgatgtcttaataggccctaaattgttctttcatttaaaaagtggcagtctctgaagtcatttgtgagcttgtatgacttttgtatttagcaatgttgcatgctcacataattgatattaaaagtaatacatttttctgaaatgtaaaaaaaaaaaaaaaaaaaaaaaaaaaaaaaaaaa

SEQ ID NO: 2 - protein, 76 aa, Sus scrofa
MTDVETTYADFIASGRTGRRNAIHDILVSSASGNSNELALKLAGLDINKTEGEEDAQRSSTEQSGEAQGEAAKSES

SEQ ID NO: 3 - DNA, 27 bp, Homo sapiens
  CDS (1)..(27)
atgtggatatttggtagcaatgactga

SEQ ID NO: 4 - DNA, 27 bp, Sus scrofa
  CDS (1)..(27)
atgtggatatttgttagcaatgactga

SEQ ID NO: 5 - protein, 8 aa, Homo sapiens
MWIFGSND

SEQ ID NO: 6 - protein, 8 aa, Sus scrofa
MWIFVSND

(19) United States
(12) Patent Application Publication
(10) Pub. No.: US 2002/0103117 A1
(43) Pub. Date: Aug. 1, 2002

(54) Use of protein kinase-inhibitor-alpha

(76) Inventor: Ralph Knoell, Friedberg, DE (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 09/773,776

(22) Filed: Feb. 2, 2001

Related U.S. Application Data
(60) Provisional application No. 60/181,927, filed on Feb. 11, 2000.

(30) Foreign Application Priority Data
Feb. 3, 2000 (DE) 10004858.7

(51) Int. Cl. A61K 48/00; A61K 38/57

(52) U.S. Cl. 514/12; 514/44

(57) ABSTRACT

In one aspect, this invention relates to the use of PKI-alpha and/or its derivatives for reduction in blood pressure, as beta-antagonists and for the production of a medication for the prevention and/or treatment of diseases in which a beta-antagonist can be used.

In another aspect, the invention relates to the use of an inhibitor of PKI-alpha or a derivative thereof for increase in blood pressure, as a beta-agonist and for the production of a medication for the treatment and/or prevention of diseases in which a beta-agonist can be used.

This invention relates to the use of protein kinase-inhibitor-alpha (PKI-alpha), the use of inhibitors of protein kinase-inhibitor-alpha, and processes for screening beta-agonists and beta-antagonists.

In 1975, the phenomenon of “myocardial stunning” was described for the first time. This refers to the property of the myocardium to exhibit a prolonged, reversible myocardial dysfunction after a brief ischemia caused by coronary occlusions, i.e., the myocardium does not show the expected contractility. This reversible condition lasts for several hours and contradicted the view previously commonly held by experts that an ischemic condition of more than 45 minutes necessarily is accompanied by necrosis of the myocytes.

As a pathogenicity mechanism for the “myocardial stunning,” the formation of oxygen radicals, calcium overloading, dysfunction of the sarcoplasmatic reticulum and troponin proteolysis was discussed (Bolli; Mechanism of Myocardial “Stunning.” Circulation 1990: 723-38; and Wijns, Vatner, Camici. Hibernating Myocardium. New England Journal of Medicine. 1998. 173-81.) Each of these mechanisms is not suitable, however, to describe the phenomenon completely (Duncker et al.; Myocardial Stunning Remaining Questions. Cardiovasc Res 1998; 38: 549-558).

The protein kinase-A that is present in the heart (hereinafter also referred to as PKA), which regulates the myocardial contractility by means of phospholamban and phosphorylation of troponin I, is controlled by receptors that are coupled to the adenylate cyclase, which in turn is regulated by catecholamines. The enzyme activity is regulated both by protein kinase-inhibitors (referred to below as PKI) and by cAMP. Two forms of protein kinase-inhibitors are found in the heart: the alpha-form and the beta-form (PKI-alpha and PKI-beta). Both act as competitive inhibitors by the Cα and Cβ subunits of the active protein kinase-A being blocked. In this case, the α-form is six times more effective than the β-form.

A protein kinase-inhibitor that is known in the prior art is the protein kinase-inhibitor-alpha (also referred to here as PKI-alpha). PKI-alpha has a length of 76 amino acids, whereby the portion of the primary sequence that is essential to the inhibitory action seems to be limited to the area of amino acids 14 to 22. This sequence is 100% identical in humans, pigs, chickens, rats and mice. On the level of mRNA, the 5′ end of the entire mRNA of pigs shows a sequence homology of about 90% compared to that of the genome of the human, the mouse, the rat and the chicken. A recombinant PKI-alpha that comprises only amino acids 14 to 22 of the complete PKI-alpha-amino acid sequence is cell-permeable and can be obtained from Calbiochem (product number #476485).

On its 5′-end, the pig cDNA that codes for PKI-alpha has a second open reader frame that codes for a polypeptide with 9 amino acids, and on its 3′-end, it has several different poly(A)-signal sequences (gene bank #bankit256111AF132737).

The object of this invention is to provide new uses for PKI-alpha and for an inhibitor of PKI-alpha.

According to the invention, the object is achieved by the use of PKI-alpha and/or its derivatives for reduction in blood pressure.

In another aspect of the invention, the object according to the invention is achieved by the use of PKI-alpha and/or its derivatives as beta-antagonist.

In yet another aspect of the invention, the object is achieved by the use of PKI-alpha and/or its derivatives for the production of a medication for the prevention and/or treatment of diseases in which a beta-antagonist can be used.

In the use according to the invention, it can be provided that the PKI-alpha comprises an amino acid sequence that is shown in SEQ ID No. 2.

In an embodiment of the uses according to the invention, the PKI-alpha is coded by a nucleic acid, which corresponds to the coding sequence of the sequence that is shown in SEQ ID No. 1 or a nucleic acid that is produced from it because of the degeneration of the genetic code or a nucleic acid that hybridizes thereto.

In the uses according to the invention, it can also be provided that the PKI-alpha is used in a shortened form.

In a preferred embodiment of the uses according to the invention, it can be provided that the PKI-alpha comprises amino acids 14 to 22 of the PKI-alpha sequence, especially the sequence of PKI-alpha according to SEQ ID No. 1 or SEQ ID No. 2.

In especially preferred embodiments of the uses according to the invention, it is provided that the PKI-alpha or its derivative is used for treating and/or preventing diseases that are selected from the group that comprises cardiac irregularities, hyperkinetic heart syndrome, angina pectoris, myocardial infarction, acute myocardial infarction, cardiac insufficiency, arterial hypertonia, essential and renal hypertonia, portal hypertension, bleeding from the esophageal varices, pheochromocytoma, overdosage of beta-sympathomimetic agents and choline receptor blockers, glaucoma simplex, hyperthyreosis, thyrotoxicosis, migraine, essential tremor, kinetic tremor and alcohol withdrawal syndrome.

In other especially preferred embodiments of the uses according to the invention, it is provided that PKI-alpha or its derivative is used for post-infarction medication, prophylaxis of recurrent infarction and/or as a sedative.

According to the invention, the object is achieved by the use of-an inhibitor of PKI-alpha or a derivative thereof for increase in blood pressure.

In another aspect, the object is achieved by the use of an inhibitor of the PKI-alpha or a derivative thereof as a beta-agonist.

In yet another aspect, the object is achieved by the use of an inhibitor of the PKI-alpha or a derivative thereof for the production of a medication for the treatment and/or prevention of diseases, in which a beta-agonist can be used.

In a preferred embodiment of the uses according to the invention, it is provided here that the inhibitor influences the transcription of the PKI-alpha.

In the use according to the invention, it can further be provided that the PKI-alpha is a PKI-alpha, as described herein.

In another embodiment of the uses according to the invention, it is provided that the inhibitor of the PKI-alpha comprises an amino acid sequence, which is selected from the group that comprises sequences with SEQ ID No. 3 and SEQ ID No. 4.

In especially preferred embodiments of the uses according to the invention, it is provided that the inhibitor of the PKI-alpha is used for treatment and/or prevention of diseases that are selected from the group that comprises cardiac insufficiency, myocardial infarction, chronic and acute cardiac insufficiency and hypotonia.

In another aspect, the object is achieved by a process according to the invention for screening agents for reduction in blood pressure, beta-antagonists and/or agents for treatment and/or prevention of diseases in which a beta-antagonist can be used, whereby it is provided that

a mixture is prepared from a beta-receptor, a beta-agonist of the beta-receptor and a candidate beta-antagonist, and

it is determined to what extent the binding behavior of the beta-agonist to the beta-receptor is changed by the candidate beta-antagonist.

In an embodiment of the process according to the invention, it is provided that the change of the binding behavior of the beta-agonist to the beta-receptor that is observed under the influence of the candidate beta-antagonist is compared to the change of the binding behavior of the beta-agonist to the beta-receptor that is observed under the influence of the beta-antagonist, as described herein.

In a preferred embodiment of the process according to the invention, it is provided that the beta-receptor is selected from the group that comprises beta1 and beta2 receptors.

In yet another embodiment of the process according to the invention, it is provided that the beta-agonist is a sympathomimetic agent, especially one that is selected from the group that comprises noradrenalin, adrenalin, dopamine and dobutamine.

Finally, it can be provided in the process according to the invention that the beta-agonist is a beta-agonist, as described herein.

In yet another aspect, the object is achieved by at process for screening agents for reduction in blood pressure, beta-agonists and/or agents for treatment and/or prevention of diseases in which a beta-agonist can be used, whereby

a transcription system is provided for a beta-antagonist,

a candidate beta-agonist is added to the transcription system, and

it is determined to what extent the transcription for the beta-antagonists is changed by the beta-agonists.

In a preferred embodiment of the process according to the invention, it is provided that the change of the transcription of the beta-antagonist that is observed under the influence of the candidate beta-agonist is compared to the change of the transcription of the beta-antagonist that is observed under the influence of the beta-agonist, as described herein.

In another embodiment of the process, it is provided that the beta-antagonist is as described herein.

In yet another embodiment of the process according to the invention, it is provided that the beta-antagonist can be obtained according to one of the processes according to the invention.

This invention is based on the surprising finding that PKI-alpha has a negatively inotropic action, i.e., an action that impairs the myocardial function. This action takes place via inhibition of protein kinase A, which is responsible for the phosphorylation of phospholamban and troponin (so-called beta-adrenergic signal transduction). Because of the negatively inotropic action of PKI-alpha, the myocardial function, especially in the case of cardiac insufficiency, is improved conversely by inhibition of the PKI-alpha.

The uses according to the invention are all based on the above-described findings that PKI-alpha has a concentration-dependent, negatively inotropic action and is involved in beta-adrenergic signal transduction. It follows from the above in one aspect that PKI-alpha can be used for the purpose of reducing blood pressure or producing a medication. In another aspect, it follows from the above that PKI-alpha can be used as a beta-antagonist. The latter lies in the fact that the action of the catecholamines that bind beta-receptors to the protein kinase-A that regulates the contractility can be counteracted by the protein kinase-inhibitors, and thus the latter show a beta-antagonistic action.

Because of the beta-antagonistic action of the PKI-alpha, the latter is also suitable for the production of medications in which in general a beta-antagonist is used. In other words: because of the beta-adrenergic action of PKI-alpha, the latter or derivatives thereof can be used instead of or in addition to the previously used beta-antagonist(s) in medications. In this case, there are basically no limitations whatsoever, so that with PKI-alpha or with a medication that contains the latter, a replacement is now provided for any medication that contains a beta-antagonist as a pharmaceutical active ingredient.

Since the PKI-alpha is expressed not only in the muscular tissue but also in the nerve tissue, especially in the brain, in particular neurophysiological indication fields for the PKI-alpha and inhibitors of the same are also within the scope of this invention. By way of example, reference can be made here to migraines for whose treatment beta-antagonists are used, which can be replaced by PKI-alpha according to the invention. For one skilled in the art, it follows that any tissue that expresses PKI-alpha and forms pathological or changed conditions can be therapeutically or preventively treated according to the invention with PKI-alpha and inhibitors thereof.

A number of advantages are created with the uses according to the invention. With the use of PKI-alpha and an inhibitor thereof, the group of pharmaceutically active compounds is considerably enlarged, especially to the extent that a completely new class of compounds is introduced into these therapeutically very important indication fields. Moreover, the use of PKI-alpha and inhibitors thereof is also advantageous to the extent that they are prepared very early in the cascade of the beta-adrenergic signal transduction and thus are not subject to the undesirable down-regulation of the beta-receptors that is associated with many drawbacks. These advantages are to be explained further below based on cardiac insufficiency.

In Western industrialized countries, cardiac insufficiency is considered to be among the most serious diseases because of its high prevalence (1-1.5%) and morbidity rate in the adult population as well as its correspondingly high costs (Sharpe, N. et al.; Lancet 352 (Suppl. I), 3-7 (1998)). A number of medications are used to treat it, only a few of which, however, are actually effective, i.e., over a period of many years. These include mainly β-blocking substances and ACE-inhibitors (Cleland, J.; Lancet 352 (Suppl. I), 1-2 (1998)). By contrast, the additionally commonly used calcium antagonists (e.g., adalat-nifedipine) are discredited (Prescrire Int 7(35), 90-91 (1998); and Ishibashi, Y. et al.; Clin Exp Pharmacol Physiol 26(5-6), 404-10 (1999)). In particular their acute effects, which seem to increase the mortality of the patients under treatment:, indicate that caution be exercised when these medications are used. By contrast, β-blockers seem to have considerable advantages, especially relative to mortality, for the patients in question. Unfortunately, these medications act via β1 and β2 receptors, which are subject to the so-called down-regulation and therefore can miss their site of action (Massie, B.; Lancet 352 (Suppl I), 29-33 (1998). Attempts are therefore increasingly made to find substance classes that have their site of action further above the β-adrenergic signal transduction cascade. By the inhibition of protein kinase-A, mainly by intracoronary administration, the PKI-alpha, especially amino acids 14-22 thereof, seems to belong to the desired substance class.

In this connection, an inhibitor of the PKI-alpha, especially the inhibitor of the PKI-alpha (PKI-alpha-I) that is coded by the second open reader frame of the PKI-alpha, therefore seems especially advantageous. As desired in each case, both the beta-antagonistic actions and the beta-agonistic actions can thus be shown off to advantage by the use of PKI-alpha or an inhibitor thereof.

The amino acid sequence of PKI-alpha is depicted in SEQ ID No. 1 together with the nucleic acid sequence that codes it and as an amino acid sequence as such in SEQ ID No. 2. For one skilled in the art, it follows that the PKI-alpha is not limited to the PKI-alpha that has the sequences that are depicted as sequences SEQ ID No. 1 and SEQ ID No. 2. Rather, a PKI-alpha here is defined as any protein kinase-inhibitor that influences the activity of protein kinase-A. In this respect, a functional definition is based on the term of protein kinase-inhibitor hereinafter.

Because of the functional definition of the term of protein kinase-inhibitor that is used here, the nucleic acid sequence that is shown in SEQ ID No. 1 is also only one example of a sequence that codes a protein kinase-inhibitor. Other sequences follow from the degeneration of the genetic code.

In its native form, the protein kinase-inhibitor-alpha has a length of 76 amino acids. Actually, the range of the primary sequence of amino acids 14 to 22 that is relevant for the inhibitory action is extended, so that even shortened (“truncated”) forms of protein kinase-inhibitors, especially of PKI-alpha, can be used according to the invention. In this case, the use of PKI-alpha, whose sequence is limited to amino acids 14 to 22 of the complete amino acid sequence, seems to be of special advantage. This shortened PKI-alpha can pass through membranes or is membrane-permeable, i.e., it can pass through a cytoplasmatic membrane into the intracellular space and there reach the intracellular compartments, especially the sarcoplasmatic reticulum and protein kinase-A. PKI-alpha and especially a shortened form and quite especially the shortened form that comprises amino acids 14 to 22 of the complete primary sequence of the PKI-alpha, as shown in SEQ ID No. 1 or SEQ ID No. 2, can be administered intracoronarily, intraperitoneally or else intravenously.

In this case, it is within the framework of the abilities of one skilled in the art that PKI-alpha, just like its inhibitor, can be present in modified form. Such a modification can be carried out, for example, in connection with the requirements of galenicals. Other modifications can pertain to pharmacological and toxicological properties.

Formulations that comprise PKI-alpha or an inhibitor thereof can comprise the commonly used galenical adjuvants and components such as pharmaceutically acceptable vehicles and buffers.

Beta-blockers, i.e., beta-antagonists, and thus PKI-alpha according to the invention and its derivatives are used, for example, as or for or in

1. antiarrhythmic agents (cardiac irregularities)

2. hyperkinetic heart syndrome

3. antianginal medications (angina pectoris)

4. improvement in the prognosis-in patients with myocardial infarction (post-infarction medication), acute myocardial infarction and prophylaxis of recurrent infarction

5. therapy in cardiac insufficiency

6. antihypertensive agents (arterial hypertonia, essential and renal hypertonia)

7. portal hypertension, bleeding from the esophageal varices

8. pheochromocytona (only usable after preceding alpha-blocking)

9. overdosage of β-sympathomimetic agents and m-cholinoceptor enceptor blockers

10. glaucoma in the eye (glaucoma simplex)

11. hyperthyreosis and thyrotoxicosis

12. therapy of central nervous diseases, such as, for example, migraine, and as a sedative (central calming effect) (essential tremor, kinetic tremor and alcohol withdrawal syndrome)

The use of an inhibitor of the PKI-alpha according to the invention (also referred to as PKI-alpha-I below hereinafter) is based in turn on the surprising finding that PKI-alpha has a concentration-dependent, ionotropic action and is involved in the beta-adrenergic signal transduction. In view of these properties of PKI-alpha, an inhibitor of PKI-alpha can result in a modification and reversal of the actions of PKI-alpha. Because of this, an inhibitor of the PKI-alpha can be used for increase in blood pressure or a medication that is suitable for this purpose. Since the inhibitor of PKI-alpha acts on an inhibitor of the beta-adrenergic signal transfer, it itself acts as a beta-agonist and can be used accordingly. This use comprises in general the use for the production of a medication for the treatment and prevention of diseases in which beta-agonists can be used: an inhibitor of PKI-alpha can thus be used in addition to the previously used beta-agonist(s) in medications. In this case, basically no limitations whatsoever exist, so that a replacement for any medication that contains a beta-agonist as a pharmaceutical active ingredient is now prepared with the inhibitor of PKI-alpha.

In this case, a functional definition is in turn based on the term of the inhibitor of PKI-alpha. An inhibitor of PKI-alpha is thus any compound that inhibits the action of PKI-alpha, whereby it is insignificant to what extent the inhibition of the PKI-alpha is carried out, to which chemical compound class the inhibitor of the PKI-alpha can be assigned and on the basis of which mechanism the inhibitor of PKI-alpha exerts its action.

A mechanism that can take as a basis the action of an inhibitor of PKI-alpha is the inhibition of the transcription of PKI-alpha. For example, the gene product of the second open reader frame of the PKI-alpha gene acts as an inhibitor in this sense. The fact that, in this case, the gene product can be the active agent seems to follow from Wang, X. et al.; Mol Pharm 54, 514-524 (1998). The amino acid sequence of this gene product of humans was

Met Trp Ile Phe Gly Ser Asn Asp and is recorded hereinafter as SEQ ID No. 3, together with the coding nucleic acid sequence.

The corresponding sequence of the pig is

Met Trp Ile Phe Val Ser Asn Asp and is recorded hereinafter as SEQ ID No. 4 together with the coding nucleic acid sequence.

The difference in form of an amino acid exchange at position 5 of the amino acid sequence that exists between the two human and pig types is based on an exchange on the level of nucleic acid, whereby position 2 of the fifth codon is changed. In the case of the sequence in humans, glycine is a fifth amino acid, while the latter is valine in the case of the pig sequence. The corresponding base exchange is a change from G (human) to T (pig).

It is obvious to one skilled in the art that deviations from the sequences that are disclosed in SEQ ID No. 3 and SEQ ID No. 4 are possible, while the derivative that is derived from the sequences still exhibits an inhibitory action for PKI-alpha, especially its transcription.

Beta-agonists and thus inhibitors of PKI-alpha, as disclosed herein, can be used for or in

1. increasing the contractility of the myocardium (desirable in the case of serious cardiac insufficiency or in myocardial infarction patients (chronic cardiac insufficiency and acute cardiac insufficiency)

2. hypotonia

It is obvious to one skilled in the art that the uses according to the invention can be employed within the framework of the therapeutic and preventative diseases, especially in individuals who require such treatment.

The processes according to the invention-are processes found or prepared with their compounds that can be used as PKI-alpha, i.e., as inhibitors of protein kinase A or as inhibitors thereof, especially in connection with one or more of the diseases or indication fields disclosed herein. In this case, the processes can also be oriented such that starting from a wide variety of compounds, one or more of them with a corresponding property are to be determined (“screening”). A compound that possibly has a corresponding property is also referred to hereinafter as a “candidate” compound.

In the process according to the invention for determining or for screening a compound that has a beta-antagonistic action in the broadest sense, the receptor that is provided can be present as an isolated molecule, for example in solution or immobilized. It is also within the scope of this invention, however, that the receptor is present in a biological system. Such a biological system can be an in vitro system or an in vivo system. A typical in vitro system in this case is a translation or transcription system. A typical in vivo system in this case is a cellular system, for example a cell, especially a myocardial cell or nerve cell that contains the genetic information for PKI-alpha. A cellular system can also be a cell, however, in which nucleic acid that preferably codes for PKI-alpha or a portion of it using genetic-engineering methods or nucleic acid that controls its expression is introduced. When using the in vivo and in vitro systems, use can also be made here of the stress inducibility of the PKI-alpha that is also disclosed herein.

In the process according to the invention, it can further be provided that the action of a candidate antagonist is evaluated on the basis of a comparison of the action of PKI-alpha and its derivatives, as they are described herein. As a result, even at a very early point of the screening or development process of novel beta-antagonists and beta-agonists, a quantification of the action of the tested compounds can be carried out. In this case, the process according to the invention can also provide that a beta-agonist, as described herein, is used.

In the process according to the invention for determining or for screening a compound that has a beta-agonistic action in the broadest sense, the beta-agonist that is provided can be present as an isolated molecule, for example in solution or immobilized. It is also within the scope of this invention, however, that the candidate agonist is present in a biological system. Such a biological system can be an in vitro system or an in vivo system. A typical in vitro system in this case is typically a cell-free translation or transcription system. A typical in vivo system in this case is a cellular system, for example a cell, especially a myocardial cell or nerve cell that contains the genetic-engineering information for the candidate beta-agonist. A cellular system can also be a cell, however, in which nucleic acid that codes for the candidate beta-agonist or a portion thereof preferably using genetic-engineering methods or nucleic acid that controls its expression is introduced.

The transcription system for a beta-antagonist that is used within the framework of the process according to the invention can be an in vivo system or an in vitro system in this case. A typical in vitro system in this case is a translation or transcription system. A typical in vivo system in this case is a cellular system, for example a cell, especially a myocardial cell or nerve cell that contains the genetic-engineering information for PKI-alpha. A cellular system can also be a cell, however, in which nucleic acid that codes for PKI-alpha or a portion thereof preferably using genetic-engineering methods or nucleic acid that controls its expression is introduced. When using the in vivo and in vitro systems, use can also be made here of the stress inducibility of the PKI-alpha that is also disclosed herein.

It is also within the framework of the process according to the invention that the transcription system for a beta-antagonist also comprises the candidate beta-agonist or the nucleic acid that codes for it or controls its expression. In addition, it is within the framework of the process according to the invention that beta-antagonists and beta-agonists, as they are disclosed and described herein or can be determined according to one of the processes disclosed herein or can be determined as such, are used therein.

In the process according to the invention, it can also be provided that the action of a candidate beta-agonist is evaluated on the basis of a comparison of the change of the transcription of the beta-antagonist that is observed under the influence of the candidate beta-agonist with the change of the transcription of the beta-antagonist that is observed under the influence of a beta-agonist, as described herein. As a result, even at a very early point of the screening or development process of novel beta-agonists, a quantification of the action of the tested compounds can be carried out.

The invention is further illustrated by the following figures, examples and the sequence protocol, from which additional features and advantages of the invention can be produced. Here:

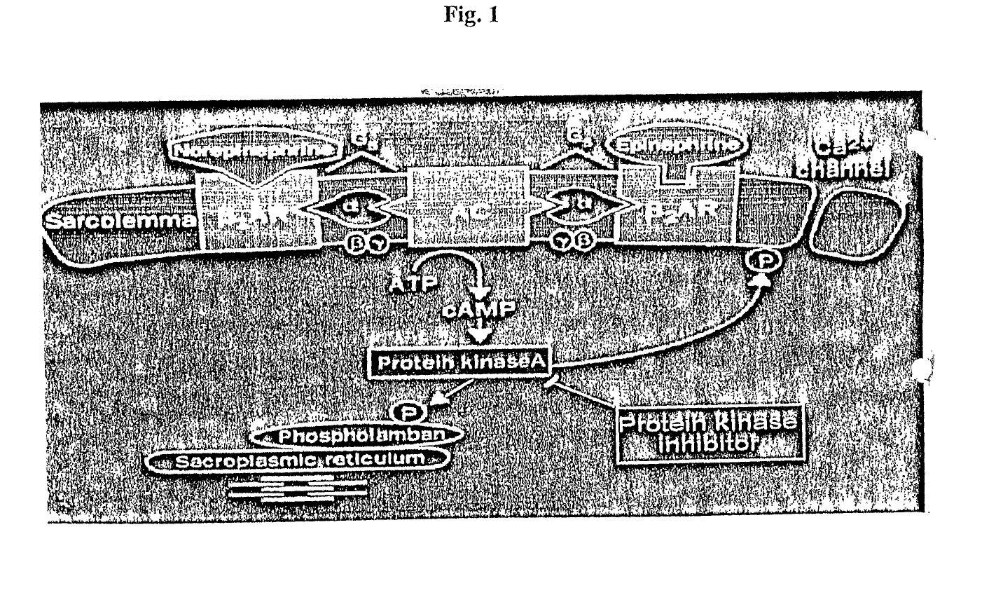

FIG. 1 shows the function of protein-kinase-A and the PKI-alpha in the beta-adrenergic signal transduction: The signal transduction cascade of the β1 and β2 receptors (β1 and β2 AR) that protein-kinases-A activate via β-, β- and y-G-protein subunits as well as the adenylate cyclase (AC) and cAMP elevations is shown in more detail. Protein-kinase-A can be inhibited by the PKI-alpha, by which the effects of protein-kinase-A can be antagonized. Protein-kinase-A phosphorylates especially phospholamban, by which the positively inotropic action can be explained.

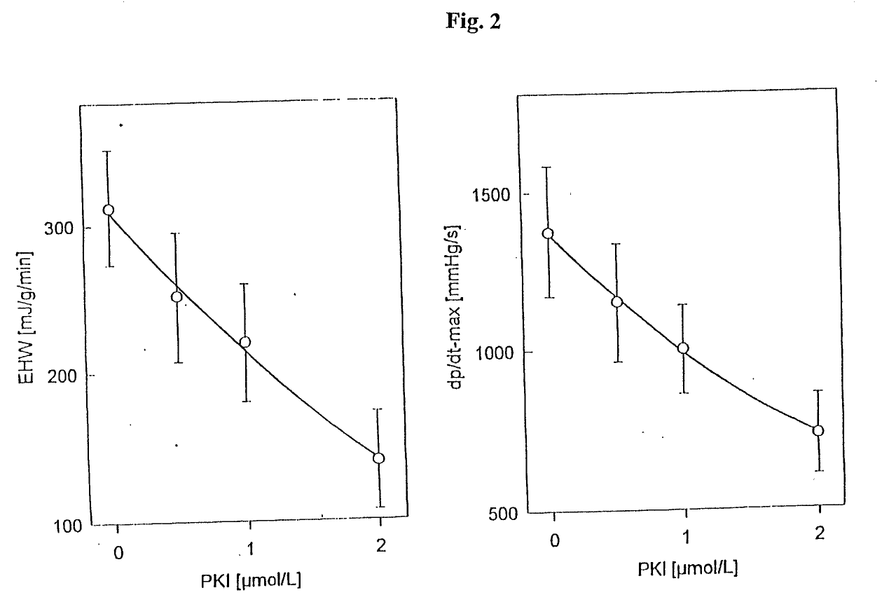

FIG. 2 shows the reduction of the function of the left ventricle after the administration of PKI-alpha that is expressed as dp/dt and external heart work (EHW).

### EXAMPLES

### Example 1

### Studies on the action of PKI-alpha on a functioning rat heart

Hearts from a total of six Sprague Dawley rats with a weight of about 300 g were isolated and perfused as described in Schulze et al. (Schulze et al.; Circulation 1990: 959-69). The hemodynamic parameters including contractility, external heart work (EHW), coronary and aortic flow were recorded about 15 minutes after antegrade perfusion. Cell-permeable, recombinant PKI-alpha (amino acids 14 to 22, Calbiochem #476485) was then administered. The compound was infused directly into the atrium canula using a motor pump. The infusion rate was continuously adapted to the emission volume (the sum of the aortic and coronary flow) and increased in five-minute intervals to reach the final PKI-alpha concentrations in the perfusate of 0.5, 1.0 and 2.0 μmol/l. The results pertaining to the external heart volume and the plot of dp/dtₘₐₓ under these test conditions are shown in FIG. 2.

The infusion of the shortened PKI-alpha caused a pronounced, reversible, concentration-dependent (up to 2 μmol) depression of the myocardial function, which was comparable to the dysfunction observed after short coronary occlusion. The measured reduction of the external heart work and dp/dt-max was significant in the paired t-test for all tested concentrations. The perfusion with the shortened PKI-alpha resulted in a reduction of the external heart work by 43% and a decrease of the left ventricular function, expressed as dp/dt-max, by 53%.

It follows from these studies that the shortened form of the PKI-alpha is suitable for reduction in blood pressure and in general for use as a new form or substance class of a beta-antagonist.

### Example 2

### Studies of Cell Cultures

The protein-kinase-inhibitor alpha shortened to amino acids 14 to 22 was added to the cell cultures of myocytes (e.g., C9H2 cells, available from the American Type Culture Collection), and the activity of protein kinase-A was determined. A dose-dependent reduction of activity was noted.

The 9-amino-acid-long PKI-alpha inhibitor (the gene product of the second open reader frame of the PKI gene in humans and in pigs) was added to the cell cultures of myocytes, and the expression of the PKI-alpha and the activity of protein kinase-A were determined. An increase of the activity of the PYA dependent on the dose of the PKI-alpha inhibitor was noted.

Cell cultures of myocytes were transfixed with an expression plasmid with the sequence of PKI-alpha, as described in, for example, Olsen (Olsen et al.; Endocrinology 5; 1246-1256 (1991)), and the expression of the PKI-alpha was determined. At the same time, the activity of protein kinase-A, which is inhibited specifically by PKI-alpha, was determined. (Protein kinase-A is contained both in striped muscle cells and in smooth muscle cells (Wang, X. et al.; Mol Pharm 54, 514-524 (1998))). Various concentrations of the protein kinase-inhibitor-alpha inhibitors (PKI-alpha-I) were added to these cell cultures. The expression of the PKI-alpha was reduced as a function of the concentration of the PKI-alpha inhibitor.

The disclosure of the various bibliographic references that are cited herein is herewith integrated by reference.

The features of the invention that are disclosed in the above description, claims and drawing can be important both individually and in any combinations for the implementation of the invention in its various embodiments.

1. Use of PKI-alpha and/or its derivatives for reduction in blood pressure.

2. Use of PKI-alpha and/or its derivatives as beta-antagonists.

3. Use of PKI-alpha and/or its derivatives for the production of a medication for the prevention and/or treatment of diseases in which a beta-antagonist can be used.

4. Use according to one of claims 1 to 3, characterized in that PKI-alpha comprises an amino acid sequence that is shown in SEQ ID No. 2.

5. Use according to one of claims 1 to 3, wherein the PKI-alpha is coded by a nucleic acid, which corresponds to the coding sequence of the sequence that is shown in SEQ ID No. 1 or a nucleic acid that is produced from it because of the degeneration of the genetic code or a nucleic acid that hybridizes thereto.

6. Use according to one of claims 1 to 5, wherein the PKI-alpha is used in a shortened form.

7. Use according to claim 6, wherein the PKI-alpha comprises amino acids 14 to 22 of the PKI-alpha sequence, especially the sequence of PKI-alpha according to SEQ ID No. 1 or SEQ ID No. 2.

8. Use according to one of claims 1 to 7, wherein the PKI-alpha is used for treatment and/or prevention of diseases that are selected from the group that comprises cardiac irregularities, hyperkinetic heart syndrome, angina pectoris, myocardial infarction, acute myocardial infarction, cardiac insufficiency, arterial hypertonia, essential and renal hypertonia, portal hypertension, bleeding from the esophageal varices, pheochromocytoma, overdosage of beta-sympathomimetic agents and cholinoreceptor blockers, glaucoma simplex, hyperthyreosis, thyrotoxicosis, migraine, essential tremor, kinetic tremor and alcohol withdrawal syndrome.

9. Use according to one of claims 1 to 8, wherein the PKI-alpha is used for post-infarction medication, prophylaxis of recurrent infarction and/or as a sedative.

10. Use of an inhibitor of PKI-alpha or a derivative thereof for increase in blood pressure.

11. Use of an inhibitor of PKI-alpha or a derivative thereof as a beta-agonist.

12. Use of an inhibitor of PKI-alpha or a derivative thereof for the production of a medication for the treatment and/or prevention of diseases in which a beta-agonist can be used.

13. Use according to one of claims 10 to 12, wherein the inhibitor influences the transcription of the PKI-alpha.

14. Use according to one of claims 10 to 13, wherein the PKI-alpha is a PKI-alpha according to one of claims 1 to 9.

15. Use according to one of claims 10 to 14, wherein the inhibitor of the PKI-alpha comprises an amino acid sequence that is selected from the group that comprises sequences with SEQ ID No. 3 and SEQ ID No. 4.

16. Use according to one of claims 10 to 15, wherein the inhibitor of the PKI-alpha is used for treatment and/or prevention of diseases that are selected from the group that comprises cardiac insufficiency, myocardial infarction, chronic and acute cardiac insufficiency, and hypotonia.

17. Process for screening agents for blood pressure reduction, beta-antagonists and/or agents for treating and/or preventing diseases in which a beta-antagonist can be used, wherein
a mixture is prepared from a beta-receptor, a beta-agonist of the beta-receptor and a candidate beta-antagonist, and
it is determined to what extent the binding behavior of the beta-agonist to the beta-receptor is changed by the candidate beta-antagonist.

18. Process according to claim 17, wherein the change of the binding behavior of the beta-agonist to the beta-receptor that is observed under the influence of the candidate beta-antagonist is compared to the change of the binding behavior of the beta-agonist to the beta-receptor that is observed under the influence of the beta-antagonist according to one of claims 1 to 9.

19. Process according to claim 17 or 18, wherein the beta-receptor is selected from the group that comprises beta1 and beta2 receptors.

20. Process according to one of claims 17 to 19, wherein the beta-agonist is a sympathomimetic agent, especially one that is selected from the group that comprises noradrenalin, adrenalin, dopamine and dobutamine.

21. Process according to one of claims 17 to 20, wherein the beta-agonist is a beta-agonist according to one of claims 10 to 16.

22. Process for screening agents for reduction in blood pressure, beta-agonists and/or agents for treatment and/or prevention of diseases in which a beta-agonist can be used, wherein
a transcription system is provided for a beta-antagonist,
a candidate beta-agonist is added to the transcription system, and
it is determined to what extent the transcription for the beta-antagonist is changed by the beta-agonist.

23. Process according to claim 22, wherein the change of the transcription of the beta-antagonist that is observed under the influence of the candidate beta-agonist is compared to the change of the transcription of the beta-antagonist that is observed under the influence of the beta-agonist according to one of claims 11 to 16.

24. Process according to claim 22 or 23, wherein the beta-antagonist is one according to one of claims 1 to 9.

25. Process according to one of claims 22 to 24, wherein the beta-antagonist can be obtained according to a process of one of claims 17 to 21.
